(19) United States
(12) Patent Application Publication
(10) Pub. No.: US 2022/0018800 A1
(43) Pub. Date: Jan. 20, 2022

(54) DEVICE FOR SIMULTANEOUS AND RAPID DETERMINATION IN SALIVA OF THE FERTILITY HORMONES ESTRADIOL, PROGESTERONE, LUTEINIZING HORMONE AND PROLACTIN

(71) Applicant: Universidad Politecnica De Cartagena, Cartagena (murcia) (ES)

(72) Inventors: Beatriz Arevalo Perez, Madrid (ES); Juan Aznar Poveda, Cartagena (murcia) (ES); Jose Francisco Beltran Sanchez, Cartagena (murcia) (ES); Susana Campuzano Ruiz, Madrid (ES); Juan Garcia Haro, Cartagena (murcia) (ES); Antonio Javier Garcia Sanchez, Cartagena (murcia) (ES); Jose Antonio Lopez Pastor, Cartagena (murcia) (ES); Jose Manuel Pingarron Carrazon, Madrid (ES); Veronica Serafin Gonzalez-Carrato, Madrid (ES); Maria Paloma Yanez-Sedeno Orive, Madrid (ES)

(21) Appl. No.: 17/366,566

(22) Filed: Jul. 2, 2021

(30) Foreign Application Priority Data
Jul. 14, 2020 (ES) P202030720

(51) Int. Cl. G01N 27/327

(52) U.S. Cl. CPC G01N 27/3277; A61B 10/0051; G01N 27/3272

(57) ABSTRACT

Device for simultaneous and rapid determination of the fertility hormones estradiol, progesterone, luteinizing hormone and prolactin in saliva comprising a biosensor (10) provided with four working electrodes (12, 13, 14, 15), an auxiliary electrode (11) and a reference electrode (16) and a single potentiostatic circuit (2) having a single channel, which shares the auxiliary and reference electrodes, a multiplexer (3) to multiplex the signals from the four working electrodes, a transimpedance amplifier (4), an analog-digital signal converter (5) and a controller (6).

### FIELD OF THE ART

The present invention is related to the field of female fertility. More specifically, the invention relates to a device for the detection and/or quantification of different hormones related to fertilization processes in saliva samples, by means of an electrochemical sensor based on the use of magnetic particles modified with neutravidin, to quickly monitor fertility and/or reproductive status processes.

### BACKGROUND OF THE INVENTION

Performing tests for the measurement of hormones is a common, necessary, and essential procedure in treatments related to female infertility. In them, it is required to measure the concentration of hormones in two of its main phases; in the phase of ovarian stimulation to extract oocytes and in the phase of preparing the patient for embryo transfer.

Among all the hormones that are controlled during fertility treatments, estradiol (17β-estradiol), progesterone (P4), luteinizing hormone (LH) and prolactin (PRL) are considered the most relevant. These hormones are indicators of the ovarian reserve and, in addition, express the level of preparation of the body of the patient undergoing fertility treatment before embryo transfer. Depending on the level of these hormones in the body of the patient in question, the medical staff is able to determine: (i) the ideal moment to perform the extraction of the ovarian reserve; (ii) the best day to carry out the embryo transfer.

To date, the usual procedure for the measurement of the aforementioned hormones is based on invasive techniques, that is, a blood sample is extracted from the patient and an analysis of it is carried out. After the precipitation of the proteins, the determination of the hormonal levels is carried out in the serum phase of the blood. For this, techniques such as gas chromatography (Havlíková et al., 2006; Burger et al., 2007; Susan S.-C et al., 2006) or high-performance liquid chromatography (HPLC) (Zhao, et al., 2010; Makin et al 2010, Gower et al., 2010; Ric̆anyová el al., 2010) are used. Although these are standard laboratory procedures, these methodologies have the following drawbacks: (i) high cost per analysis, (ii) need for qualified personnel, (iii) long analysis time, (iv) high number of reagents required for each analysis and, (v) notable cost of the equipment required to carry out the analytics.

In addition to these drawbacks, there is an additional problem regarding the frequency of performing such analyzes. As it is an invasive technique that requires a blood draw and due to the stress that many of the patients are subjected to during assisted reproduction treatments, in most cases the medical staff only makes a weekly determination of the hormonal levels, since these biomarkers can vary in situations of stress and anxiety. This periodicity is lower than that desired by the physicians responsible for the treatments; a daily control (or even several determinations a day), would allow achieving greater precision on when to carry out the extraction of the ovarian reserve, as well as on the most exact indication of the ideal day to carry out the embryo transfer.

Due to the drawbacks of traditional determination methods and the current needs of fertility treatments, the scientific community is working on new non-invasive, faster, more sensitive, and selective procedures for the determination of the hormones of interest. Electrochemical immunosensors have a series of advantages, since they can be used in a simple way to simultaneously detect multiple target compounds in samples of highly variable nature and complexity, in addition, they also offer other added value characteristics such as high sensitivity, use of small sample volumes and quick response. In addition, additional low requirements of low cost or affordable instrumentation make them very suitable for designing compact and portable devices for use in decentralized and/or resource limited environments.

A portable electrochemical system for the detection of hormones is the one proposed by the same authors of this application in the publication “Enhanced determination of fertility hormones in saliva at disposable immunosensing platforms using a custom designed field-portable dual potentiostat Sensors and Actuators, V. Serafin, B. Arévalo, G. Martínez-García, J. Aznar-Poveda, J. A. Lopez-Pastor, J. F. Beltrán-Sánchez, A. J. Garcia-Sanchez, J. Garcia-Haro, S. Campuzano, P. Yáñez-Sedeño, J. M. Pingarrón, B 299 (2019) 126934”. This system allows the determination of two hormones simultaneously. This publication indicates that there can be used as many potentiostatic circuits based on the LMP91000 chip as measurement channels and thus quantify each of the required hormones. In the design proposed in this publication, the LMP91000 chip is used, a component specially designed to carry out potentiostatic measurements in a single channel, sharing the auxiliary and reference electrodes of one of the chips with all the necessary working electrodes of the rest of the chips (as many as substances to be measured). This configuration gives rise to undesired effects: (i) the inability to supply enough current for several simultaneous amperometries; (ii) the flow of a high current through one of the potentiostats and not through the rest produces substantial changes in temperature that result in a loss of sensitivity. Furthermore, measuring the concentration of more than two hormones with this device becomes impossible, since the wide range of currents of the different amperometries that share the same electrodes would interfere with each other, modifying the voltage at the reference electrode. The result of these effects would result in inaccurate measurements along with a notable loss of equipment precision.

### SUMMARY OF THE INVENTION

The object of the present invention is to provide a device capable of detecting the four hormones characteristic of the fertile cycle by means of a small volume of saliva maintaining reliability and with a compact equipment. To detect estradiol, progesterone, luteinizing hormone and prolactin, the invention proposes a device provided with a biosensor with four working electrodes, a reference one and an auxiliary one, also having the means to allow multiplexing of circuits. The device is provided with a single, single-channel potentiostatic circuit, sharing the auxiliary and reference electrodes, and means for multiplexing the four working electrodes on the same channel. Advantageously, the multiplexing process and times according to each of the hormones under study and the current expected to be measured in them are optimized. In this sense, program means associated with a controller calculate the optimal times that allow: (i) the stabilization of each current (ensuring the minimum time for the control amplifier to act in the event of a change in current together with the delay in stabilization introduced by the biosensor itself, in the order of tens of milliseconds); (ii) an adequate sampling frequency (ensuring a frequency that allows measuring time variation for each of the hormones in the order of tens of milliseconds) and, (iii) the time that ensures that the non-multiplexed signal is not affected by the interruption of the flow of current in the unsampled electrodes.

### BRIEF DESCRIPTION OF THE FIGURES

To help a better understanding of the features of the invention and to complement this description, the following figures are attached as an integral part thereof, the nature of which is illustrative and not limiting:

FIG. 1 shows a diagram of the device for measuring hormones, object of the present invention.

FIG. 2 is a detail of the biosensor according to the invention.

### DETAILED DESCRIPTION

Electrochemical detection is based on the modification of magnetic particles (MBs) of nano or micro-metric size (Ø=10 nm−10 μm) with neutravidin, which allows the immobilization of biomolecules on the modified magnetic particles. By applying the appropriate immunoassay format for each biomolecule, the selected immunoreagents are immobilized on the surface of these magnetic particles for the determination of each hormone. The immunoassay formats can be: i) sandwich type, wherein two different antibodies are used that recognize different epitopes in the antigen to be determined, ii) direct competitive, the hormone to be determined and the enzyme-labeled hormone are made to compete for the limited binding centers of the capture antibody, and iii) indirect competitive, the synthetic hormone is immobilized on the surface of the magnetic particles and an excess of previously labeled antibody bound to the antigen of interest is added.

To carry out electrochemical transduction, the modified MBs are resuspended in 5 μL of 0.05 M phosphate buffer of pH 6.0. This solution is then deposited on the work surface of a carbon screen-printed electrode previously placed on a housing containing an encapsulated neodymium magnet to ensure its stable and reproducible magnetic capture.

Once the magnetically modified MBs have been captured, the electrochemical detection is carried out by amperometry in the presence of hydroquinone (HQ) as a redox mediator and hydrogen peroxide (H2O2) as an enzyme substrate. Regeneration of the reduced form of the enzyme occurs through the reduced form of hydroquinone (HQred). The oxidized form of hydroquinone (HQoxy) is electrochemically reduced when the applied potential is more negative than the formal potential of the HQred/HQoxy redox pair.

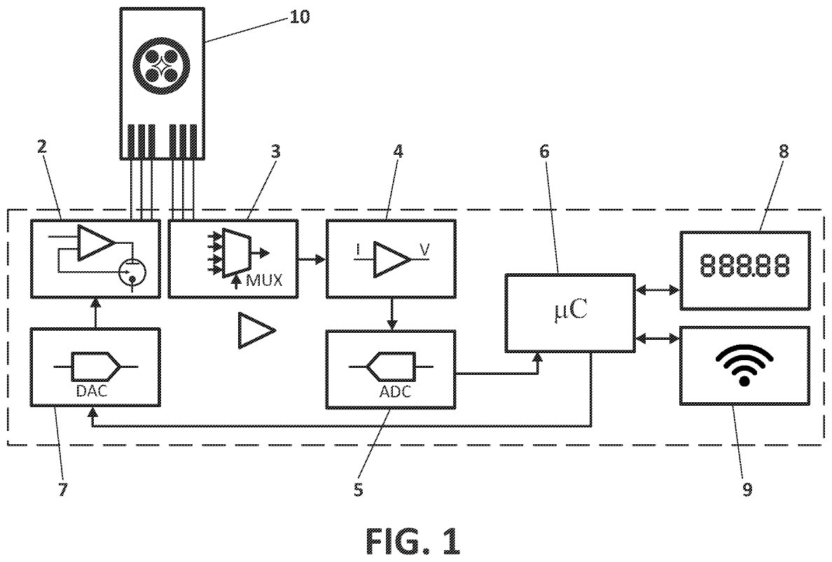

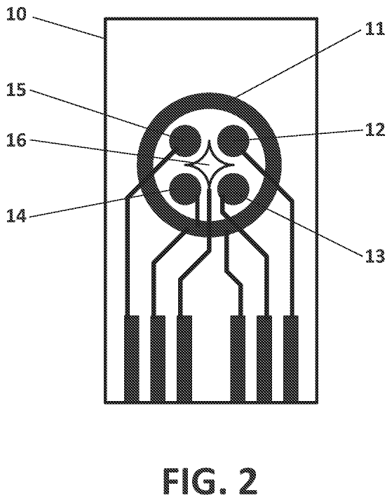

With reference to FIGS. 1 and 2, the device designed for the determination of the hormones of the present invention comprises a biosensor (10), four working electrodes (12-15), an auxiliary electrode (11) and a reference electrode (16) that are printed on a ceramic surface. The magnetic particles modified with neutravidin and with the appropriate immune complexes for each target hormone are immobilized, as previously described, with the help of a magnet on the working electrodes.

The device allows two different working modes: i) applying a linear sweep of different potentials or, ii) applying a fixed potential over time. By selecting the latter mode, a constant potential between 0 and −0.5 V is applied, for example of −0.2 V, obtaining the reading of the variation of the cathodic current generated by the enzymatic reduction reaction in each working electrode. The magnitude of the cathodic current produced will be proportional to the concentration of each hormone present in the saliva sample analyzed.

Particularly, the analysis method consists in place a drop of the appropriate volume of pretreated saliva on the surface of the working electrode, on which the modified MBs have previously been placed. The hydroquinone will cover the reference (16), auxiliary (11) electrodes and the 4 working electrodes (12, 13, 14, 15). For placing the drop, a configurable micropipette with said quantity or any other system that allows obtaining said exact quantity of liquid can be used.

For the device to work correctly and accurately, the calibration lines for each hormone to be analyzed have been previously obtained with appropriate standards. That is, in the memory of the controller (6) the calibration line is preloaded for each of the hormones to be measured (estradiol, progesterone, luteinizing hormone and prolactin). By virtue of these calibration lines, which are previously stored or loaded in the device, the concentrations of these hormones can be determined from the measured current value.

When the modified MBs have been placed on the surface of the biosensor (10) and have been captured by the encapsulated neodymium magnet on the working electrodes (12-15), a controller (6) begins to execute an amperometry process at a fixed potential in a range of 0 to −500 mV against a reference electrode made of, for example, silver (Ag), although a silver/silver chloride (Ag/AgCl) electrode or a saturated calomel electrode (SCE) can also be used. The amperometric voltage is sent from the controller (6) to the digital-analog converter (7) which acts as a generator of the voltage function to be applied in the potentiostatic circuit (2).

In the potentiostatic circuit (2) the amperometric voltage is applied between the working (12, 13, 14, 15) and reference (16) electrodes, giving rise to the reduction reaction, which generates a current to be measured. The generated current is converted to a voltage value with a transimpedance amplifier (4) and this value is in turn acquired by the controller (6) through the analog-digital converter (5).

As a single channel is used for reading the different currents associated with each hormone, it is necessary to configure the current measurement electronics, dynamically varying the working ranges of the transimpedance amplifier and the analog-digital converter (5), so that it works accurately enough.

In the device of the present invention, the electronics for stimulating and reading the signals corresponding to the four hormones have been combined in a single channel, using a single potentiostatic circuit and transimpedance amplifier, which shares the auxiliary and reference electrodes. For this, an electronic multiplexer (3) is used that directs the currents that circulate through each of the working electrodes to the transimpedance amplifier (4) and the signal reading stage and multiplexes the signal from the four electrodes on a single channel, alternating between them to measure the four current values quasi-simultaneously.

The potentiostatic circuit (2) works by keeping the potential of the working electrode at a constant level with respect to the potential of the reference electrode (16) by adjusting the current in the auxiliary electrode (11). The potentiostatic circuit (2) must maintain the same potential for the 4 hormones to be measured (between 0V and −0.5V); each of them needs a different current range, limited between hundreds of nano amps to tens of milliamps, to operate at the established potential. It is necessary to configure the full scale voltage range and the reading times of the transimpedance amplifier (4) and the analog-digital converter (5). For this, the controller (6) performs the calculations of the working ranges in real time and iteratively, in order to adjust the current values with sufficient precision according to the hormone to be determined.

In this sense, the controller (6) calculates the optimal switching times (of the order of milliseconds) between the multiplexed signals in order to: (i) stabilize each current after each change (ensuring the minimum time for the control amplifier to actuate before a change in current together with the stabilization delay introduced by the biosensor itself), (ii) compute the sampling frequency that allows measuring the variation of each of the hormones and, (iii) that the non-multiplexed signal is not affected by the interruption of current flow on unsampled electrodes.

Measurement results will be displayed on a graphical interface (8) and/or saved in an internal memory. A wireless connectivity interface (9) that can include Bluetooth, WiFi, LoRa or ZigBee protocols, will allow establishing connections with remote servers, where to save and consult the present value or the history of the results, as well as to establish connections with portable devices such as smartphones or tablets where results can be checked without the need for an additional physical screen or process can be controlled.

In view of this description and figures, the person skilled in the art will be able to understand that the invention has been described according to some preferred embodiments thereof, but that multiple variations can be introduced in said preferred embodiments.

1. Device for simultaneous and rapid determination of the fertility hormones estradiol, progesterone, luteinizing hormone and prolactin in saliva comprising a biosensor provided with four working electrodes, an auxiliary electrode and a reference electrode, characterized in that it comprises a single potentiostatic circuit having a single channel, which shares the auxiliary and reference electrodes, a multiplexer to multiplex the signals from the four working electrodes, a transimpedance amplifier, an analog-digital signal converter and a controller.

2. The device for simultaneous detection of hormones according to claim 1, wherein the controller is adapted to calculate the minimum switching time between the multiplexed signals.

3. The device for simultaneous detection of hormones according to claim 1 comprising a wireless connectivity interface that allows establishing connections with remote servers where to save and consult the results of the measurement and/or establish connections with portable devices.

4. The device for simultaneous detection of hormones according to claim 2 comprising a wireless connectivity interface that allows establishing connections with remote servers where to save and consult the results of the measurement and/or establish connections with portable devices.
